(19) United States
(12) Patent Application Publication
(10) Pub. No.: US 2025/0249240 A1
(43) Pub. Date: Aug. 7, 2025

(54) SYSTEMS AND METHODS FOR INDUCING MUSCLE CONTRACTION

(71) Applicant: Asayena, Inc., La Jolla, CA (US)

(72) Inventors: Ronald SAHYOUNI, Mission Viefo, CA (US); Mickey Ellis Abraham, La Jolla, CA (US); Herbert Tsvi Goldenberg, La Jolla, CA (US)

(73) Assignee: Asayena, Inc., La Jolla, CA (US)

(21) Appl. No.: 18/431,347

(22) Filed: Feb. 2, 2024

Related U.S. Application Data
(63) Continuation of application No. PCT/US22/39202, filed on Aug. 2, 2022.
(60) Provisional application No. 63/228,754, filed on Aug. 3, 2021.

(51) Int. Cl. A61N 1/36; A61N 1/05

(52) U.S. Cl. CPC A61N 1/36003; A61N 1/0556; A61N 1/3606; A61N 1/36139; A61N 1/36157; A61N 1/36178

(57) ABSTRACT

Apparatus for actuating a target muscle of a patient include an electromyography (EMG), a multichannel cuff electrode (MCE), and a pulse generator having both an external component and an implantable component. The EMG receives weak input from the target muscle upon flexion, and the MCE can be implanted on or in a motor nerve that innervates the target muscle. The pulse generator drives a current between two or more channels of the MCE to induce flexion of the target muscle.

### CROSS-REFERENCE TO RELATED APPLICATIONS

This application is a continuation of PCT Application No. PCT/US22/39202 (Attorney docket No. 61435-703.601), filed Aug. 2, 2022, which claims the benefit of U.S. Provisional No. 63/228,754, (Attorney Docket No. 61435-703.101), filed Aug. 3, 2021, the entire content of which is incorporated herein by reference.

### BACKGROUND

### 1. Field of the Invention

The present invention relates generally to medical devices and methods, and more particularly to methods, apparatus and systems for inducing flexion in target muscles.

Damage to movement-controlling areas of the brain and spinal cord (central nervous system, “CNS”) results in weakness and paralysis. Signals sent from the injured CNS are too compromised to communicate with the working peripheral nervous system (PNS). Without this working connection, the PNS cannot initiate movement. Stroke is one of the most common CNS injuries, leaving approximately 40 million post-stroke patients internationally with residual limb deficits. This number grows by 8.3 to 9.4 million annually. Functional improvement of these deficits is currently predicated on extensive rehabilitation, on an order of months to years. However, gains plateau at six months. A total of 50% of all stroke sur-vivors with limb deficits will be unable to meaningfully use their arm for activities of daily living. Other CNS damaging conditions also leave residual limb deficits, including spinal cord injury (27.3% of all US paralysis), multiple sclerosis, MS (18.6%), and cerebral palsy CP (8.3%). Post-stroke recovery is a minimally infiltrated market, currently with no approved invasive technological adjuvants for limb reanimation.

To date, there are few if any devices that can adequately treat such patients. A group in France recently used a multi-cuff electrode (MCE) wrapped around the radial and median nerves in the forearm to selectively stimulate muscle groups in the upper limb during intraoperative testing. See, Tigra et al., cited below. The Tigra disclosure (1) is focused on distal (i.e., forearm) without clear goal for proximal movement, (2) is neither a functional or implantable clinical device, (3) uses a single cuff system, thus cannot selectively activate nerve fascicles, (4) is limited to 4 contacts per ring electrode, (5) is not a closed loop device, 6) lacks chronic data, and (7) is targeted towards spinal cord injury patients with tetraplegia without clear translation to the stroke population.

Polasek et al. (2009) IEEE Trans Neural Syst Rehabil Eng 17(5): 428-437 describes use of multichannel spiral cuff electrodes to stimulate human nerves to elicit muscle contraction. The electrodes were percutaneously accessed or fully implanted. An open loop system was used to detect volitional movement and subsequently stimulate a nerve to restore limb function.

For these reasons, it would be desirable to provide improved apparatus, systems and methods for inducing flexion or other movement in target muscles. In particular, it would be beneficial to provide apparatus, systems and methods which minimize or eliminate the need for transcutaneous access to implantable components of the system for recharging, reprogramming, upgrading and other communication and power management tasks that arise after initial implantation.

### 2. Listing of the Background Art

Relevant art includes Polasek et al. (2009) IEEE Trans Neural Syst Rehabil Eng 17(5): 428-437; Tigra et al. (2020) J Neuroeng Rehabil 17:66; WO2002/087683; WO2019/046547; U.S. Ser. No. 11/331,493; U.S. Pat. No. 9,272,139; U.S. Ser. No. 10/751,532; US2010/031230; U.S. Ser. No. 19/293,151; US2004/0024439; U.S. Pat. Nos. 8,942,824; 9,114,246; 7,324,853; 4,750,499; US2019/0183472; and U.S. Pat. No. 9,895,546.

### SUMMARY OF THE INVENTION

The present invention comprises a closed-loop device that bypasses the injured CNS, restoring function to the weakened extremity by directly stimulating the functional nerves in the PNS. The CNS attempts to initiate arm flexion, sending a weakened signal to the PNS. An implanted or external electromyography (EMG) electrode detects this signal, directly stimulating a pulse generator connected to a multi-cuff electrode (MCE) or other electrode assembly that has been surgically implanted around one or more intact nerves of the arm (i.e., musculocutaneous nerve) or other nerve. Each electrode assembly stimulates a unique subpopulation of nerve fascicles, selectively activating discrete muscle(s), for example in the limb (i.e., the biceps brachii muscle). The arm flexes in response. In specific implementations, apparatus of the present invention (1) provide eight or more channels per ring electrode for specialized current steering and stimulation selectivity, (2) utilize a closed-loop circuit to facilitate ease-of-use for the end user, (3) allow for implantation of numerous cuffs around multiple nerves, thereby providing partial or complete reanimation of the entire arm and restoration of arm function.

While EMG electrodes are described, it will be appreciated that the systems and methods of the present invention are operable with any electrode or other sensor capable of detecting muscle movement or signals which might induce such muscle movement, including electroencephalogram (EEG), electroneurography (ENG), and the like. Additionally, the pulse generators may be controlled by external or internal drivers which are programmed to initiate muscle movement in response to a programmed pattern, e.g., for rehabilitation, or in response to a patient or other user input, e.g., the patient may have an interface allowing the patient to initiate a muscle movement on command.

Devices according to the present invention may target post-stroke recovery as a subset of the stroke management market for patients with arm weakness or paralysis. In the United States, there are approximately 7 million post-stroke patients. A total of 50-88% will have chronic upper extremity disability after aggressive rehabilitation, leading to a current market share of 3.5-6 million patients who can currently benefit from our device. The annual incidence of stroke in the US is 610,000 new cases/year, leading to additional market share of 305,000-536,000 new patients who can benefit from our device annually. In Europe, there are approximately 9 million living post-stroke patients. The annual incidence of stroke in Europe is 825,000 new cases/year, leading to 412,000-726,000 new patients who can benefit from our device annually.

In particular, the devices and systems of the present invention will be useful for treating acute or chronic stroke patients experiencing difficulty flexing their arm at the elbow but who are otherwise able to close and open their hand as well as patients recovering from spinal cord injury, traumatic brain injury, traumatic nerve injury, amongst other pathologies that affect the CNS. For example, In the United States, there are approximately 294,000 living with SCI, with an estimated 17,800 new cases annually. In Europe, there are approximately 500,000 living with SCI, with an estimated 8,900 new cases annually.

Specific devices in accordance with the principles of the present invention utilize electromyography (EMG) input to trigger an internal pulse generator (IPG) to stimulate a multichannel cuff electrode (MCE) around peripheral nerves to restore motion. For example, restoration of elbow flexion in a stroke patient with residual weakness: an EMG lead is implanted or placed near the weakened arm flexors (e.g., biceps brachii or coracobrachialis muscle) and its weak motion will trigger a multichannel cuff electrode around the musculocutaneous nerve to restore full elbow flexion strength.

In specific instances, the present invention provides closed loop devices to restore motor function in weakness or paralysis typically comprise 1) input (e.g., EMG —wired/wireless, EEG—wired/wireless, ENG-wired/wireless or other input), 2) a pulse generator that receives input, 3) output in the form of one or more cuff or other electrodes comprising one or more channels wrapped around one or more nerves.

Devices according to the present invention typically comprise one or more electrodes that can interface with a nerve. The devices may further comprise a receiver stimulator, a grounding electrode, and other components, and the devices typically have the capacity to receive EMG, ENG, or EEG information, either wirelessly or wired. The devices of the present invention are useful for treating or reducing paralysis or weakness, and methods using the device comprise (a) implanting the device in a paralyzed limb, wherein the plurality of electrodes are wrapped around a nerve (e.g., musculocutaneous nerve); (b) placing an epidermal or intramuscular electronic device in a muscle or EEG data, which serves as input when it detects muscle contraction or EEG activity or receiving ENG input from an additional electrode or the stimulation lead with input and output capability; and (c) providing a programmed and graded stimulus to the appropriate electrode, and thereby generating a symmetric contraction of the corresponding muscle on the paralyzed side.

In some embodiments, the devices of the present invention aid in achieving dynamic and spontaneous movement in a patient afflicted with weakness or paralysis of a limb.

In some embodiments, a method of treating paralysis and/or weakness in a subject, comprises (a) detecting EMG activity in a muscle, EEG activity or ENG (electroneurography) activity, and (b) treating paralysis by a direct targeted stimulation of corresponding muscles or nerve fibers on a weak or paralyzed limb.

In some embodiments, a method of treating paralysis and/or weakness or damage in a subject comprises (a) detecting the level of contraction of individual muscles or EEG signal or ENG signal; and (b) treating paralysis by a direct targeted stimulation of the corresponding nerve fibers of the paralyzed limb.

In certain embodiments, a closed loop device restores motor function in weakness or paralysis. An external pulse generator (EPG) uses electromyography (EMG) signal to wired or wirelessly pick up weak muscle activity. It converts the EMG signal into a digital signal, processes and encodes the digital signal into a radio frequency (RF) signal and sends it to the receiver-stimulator coil (RSC). A hermetically sealed stimulator contains active electronic circuits that derive power from the RF signal, decode the signal, convert it into electric currents, and send them along a multichannel cuff electrode (MCE) wrapped around a nerve. The electrodes at the end of the wire stimulate peripheral nerves to restore motion in patients with weakness or paralysis.

In other embodiments, the system may have a single cuff electrode with input and output capability that can detect weak nerve pulses and then stimulate the nerve to induce contraction or flexion of the muscle. Such a cuff electrode will be configured to have both sensing and stimulating capabilities.

In a first specific aspect of the present invention, apparatus for actuating a target muscle of a patient comprises an electrode assembly and a pulse generator including an external component and an implantable component. The electrode assembly is typically configured to be implanted on or into a motor nerve that innervates the target muscle, and the pulse generator may be configured to drive a current to the electrode assembly, typically between two or more channels of the assembly to induce flexion of the target muscle. In some instances, however, the electrode assembly could comprise, consist essentially of, or consist of a single ring or other electrode configured to stimulate the target nerve to induce muscle flexion or other movement.

The external component of the pulse generator typically comprises (1) circuitry configured to receive the flexion signal generated by the EMG and to transcutaneously deliver a stimulation signal to the implantable component and (2) a power supply configured to inductively deliver power to the implantable component. The internal component of the pulse generator typically comprises (1) circuitry configured to receive the stimulation signal from the external component and to deliver a stimulation current to the electrode and (2) a power supply configured to inductively receive power to the external component.

The apparatus optionally further comprises a sensor or driver to initiate generation of the stimulation current by the pulse generator. Most commonly, the apparatus will comprise a sensor which detects an incipient target muscle flexion or movement, typically an EMG sensor configured to be externally mounted over the target muscle to detect attempted flexion of the target muscle and generate a flexion signal in response thereto, typically being a patch electrode. Other suitable sensors include electroencephalography (EEG), electroneurography (ENG) and the like, which can detect activity in the patient's brain, or the patient's peripheral or central nerves, indicative of attempted or incipient muscle flexion or movement.

In still other instances, the apparatus may further comprise a driver programmed to stimulate the target muscle in a preselected pattern, typically for therapy. The preselected pattern can be repetitive, for example for therapy, or could be provided to allow the patient to initiate muscle movement on demand, for example using manual, verbal, or other input signals.

In exemplary embodiments, the electrode assembly comprises a cuff electrode configured to be wrapped around the motor nerve, for example comprising a multichannel cuff electrode (MCE). The MCE will usually comprise a sleeve or other backing having a two-dimensional array of electrode elements formed over an inside surface thereof configured to contact an exterior surface of the nerve when the sleeve is wrapped around or otherwise implanted over the nerve. The electrode array may comprise from two to 50 individual electrode elements, usually being from two to 20 individual electrode elements, and typical being from eight to 12 individual electrode elements. The individual electrode elements will usually have an exposed contact area (i.e., the area which contacts the outer nerve surface when the sleeve is wrapped around the nerve) in a range from 0.001 mm² to 0.1 mm², usually from 0.005 mm² to 0.05 mm², and most often being about 0.1 mm² (typically being 0.1 mm×0.1 mm).

Individual electrode elements in such electrode arrays will usually be arranged in a rectilinear pattern (disposed along axial and circumferential lines when the sleeve is wrapped around or otherwise implanted over the nerve) but could also be arranged in a spiral, irregular or other pattern. The individual electrode elements will typically be connectable to the pulse generator by individual wires in a lead, although multiplexed and/or wireless connection could be provided in some instances.

In other embodiments, the electrode assembly may comprise one, two, or more circumferential ring electrodes having a width in an axial direction a range from 0.1 mm to 0.5 mm and an inner diameter (when disposed about the nerve) in a range from 0.2 cm to 2 cm, usually from 0.1 cm to 1 cm, and most often about 0.4 cm to 0.6 cm.

In an illustrated embodiment, the MCE comprises at least four circumferentially distributed active electrode elements arranged in an annular pattern when the cuff is wrapped around the nerve, usually comprising at least two axially separated annular structures of four or more electrode elements. Additionally or alternatively, the MCE, may further comprise one or more active ring electrode elements and/or at least one ground electrode element.

In further instances, the circuitry of the external component of the pulse generator may comprise an amplifier for receiving an input signal from the sensor or driver, e.g., the flexion signal from the EMG signal, signal processing circuitry for generating the stimulation signal, and a transmitter for transcutaneously delivering the stimulation signal to the implantable component. Often, the power supply of the external component of the pulse generator comprises a rechargeable battery and an inductive coil for delivering power to the implantable component.

In a second specific aspect of the present invention, a method for inducing flexion or other movement in a target muscle of a patient comprises generating a motion or drive signal, e.g., externally generating a flexion signal in response to an electromyography (EMG) signal characteristic of attempted flexion of the target muscle receiving the flexion signal in an external component of a pulse generator. A stimulation signal generated by the external component in response to the flexion signal is wirelessly transmitted to a subcutaneously implanted internal component of the pulse generator, and a stimulation current produced by the internal component of the pulse generator is delivering to a motor nerve that innervates the target muscle to induce flexion said muscle.

Alternatively, the methods may further comprise stimulating the target muscle in a preselected pattern, typically for therapy. The preselected pattern can be repetitive, for example for therapy, or could be provided to allow the patient to initiate muscle movement on demand, for example using manual, verbal, or other input signals.

The target muscle often comprises a patient's biceps muscle wherein the target nerve comprises a musculocutaneous nerve controlling the biceps, but the methods and devices herein can also be used with a variety of other target muscles and target nerves which innervate and control the target muscles. Other target muscles include those innervated by a cranial nerve, such as the trapezius nerve.

In exemplary embodiments of the methods herein, the flexion signal is produced by a patch electrode located over the target muscle, where the patch electrode may be wired or wirelessly connected to the external component of the pulse generator.

In specific embodiments, the stimulation signal may be at least partially digital while in other embodiments the stimulation signal may be at least partially analog.

In the illustrated embodiments, the stimulation current is delivered to the motor nerve by a cuff electrode wrapped around the motor nerve, but other electrode interfaces could also be used. Cuff electrode will typically be subcutaneously wired to the implanted component of the pulse generator.

The methods herein will usually further comprise inductively recharging a battery in the implantable component of the pulse generator using a power supply in the external component, and in some instances may further comprise magnetically securing the external component of the pulse generator over the implanted component of the pulse generator.

### BRIEF DESCRIPTION OF THE DRAWINGS

FIG. 1 illustrates a first system constructed in accordance with the principles of the present invention.

FIG. 2 illustrates a second system constructed in accordance with the principles of the present invention.

FIG. 3 provides images of a prototype MCE having an 8-channel MCE including two parallel electrode rings, each having four rectangular platinum electrodes arranged 90 degrees apart within a silicone enclosure (Panel A); Intra-operative image of the two MCEs implanted at the upper and lower FN branches of Cat (Panel B); and an image of the male Omnetics connector originating from the MCE in (Panel C).

FIG. 4 is an image of a (Left) 8-channel MCE with 2 electrode “rings”, each with 4 rectangular (1.5×0.25×.038 mm) platinum electrodes in a silicon enclosure 90° apart. The current source is controlled by an 8-channel digital-to-analog converter (TDT RX8) delivers biphasic electrical 82 μs pulses. (Right) Intraoperative image of implanted MCE.

FIG. 5 illustrates apparatus according to the present invention including an electrode assembly and a pulse generator comprising an external component and an implantable component.

FIG. 6 is a rolled-out view of the electrode assembly of FIG. 5 showing an arrangement of individual electrode elements.

FIG. 7 is a block diagram showing the circuitry in the external and implantable components of the pulse generator of FIG. 5.

### DETAILED DESCRIPTION OF THE INVENTION

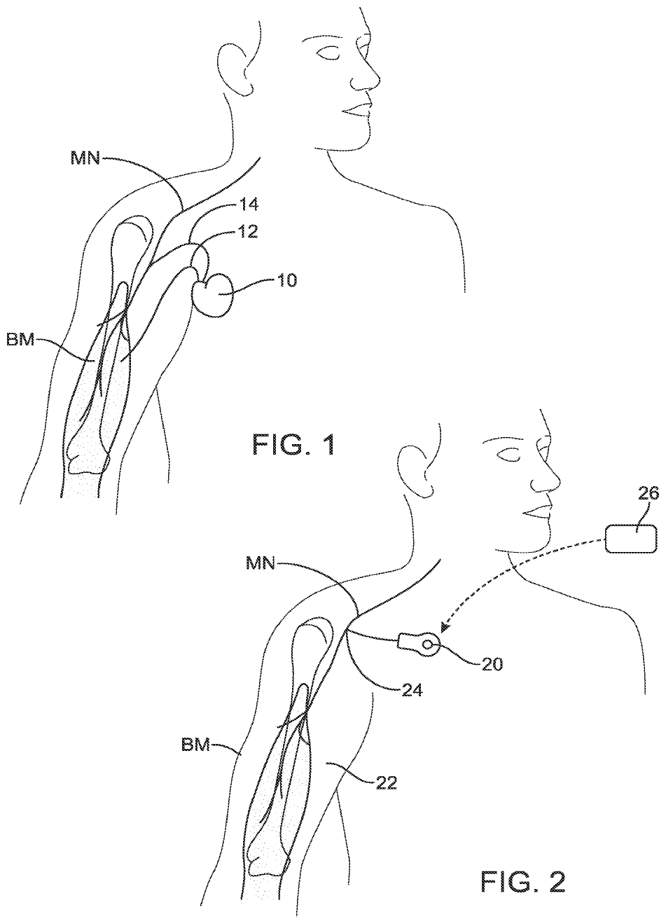

Referring to FIG. 1, a system of the present invention includes an implantable pulse generator (IPG) 10 and an implantable sensing lead 12. The IPG contains electronics and battery inside titanium case. A surgeon implants the IPG subcutaneously below the clavicle in the upper chest or in the axilla and connects the IPG to the sensing lead and a stimulation connected to a multichannel cuff electrode (MCE) 14. An algorithm detects weak electromyography (EMG) signals when arm flexion is attempted and delivers stimulation current to one or more channels of the MCE 14 wrapped around a musculocutaneous nerve MN which innervates the patient's bicep muscles BM. The IPG will typically be MRI compatible and have the ability to be wirelessly charged via a transcutaneous magnetic charging coil. The multi-channel cuff electrode (MCE) 14 has a 2-mm to 6-mm diameter and a 1-cm to 2-cm length cuff. The cuff electrode contacts are arranged in two or more “rings” with reach ring containing four individual 2-mm×1-mm rectangular (tripolar) platinum or 90/10 platinum/iridium contacts embedded in silicone positioned at 0, 90, 180, and 270 degrees around the ring. Spacing from 1-mm to 5-mm is maintained between contacts with 1-mm space from contact to cuff edge. This arrangement allows for monopolar stimulation of discrete neural locations as well as bipolar stimulation between two contacts. These dimensions are meant to be exemplary but are not meant to be limiting in any way.

The cuff can have embedded sutures in the silicone to facilitate cuff placement. The surgeon positions the MCE around a patient's musculocutaneous nerve and connects the connector tip end of the lead to the IPG. The cuff electrodes apply electrical current that stimulates the musculocutaneous nerve which causes the arm to flex at the elbow. The sensing lead may be placed in the biceps brachii muscle and contains a electromyography (EMG) sensor for detecting weak EMG signals during attempted arm flexion.

The IPG is configured to wirelessly interface with an external handheld device. The handheld device may be placed on the skin over the implant to provide a non-invasive means for the patient to activate the IPG, to adjust the stimulation parameters (within the physician prescribed limits), to check battery status, and to optionally charge wirelessly.

The IPG is further configured to wirelessly interface with a physician programmer which may comprise a tablet computer and a telemetry cable having a telemetry head. The telemetry head communicates with the IPG through the skin via short-range radiofrequency (RF) telemetry. Telemetry communication allows the physician to non-invasively interrogate and configure the IPG settings. The physician programmer has the capability to monitor EMG waveforms, configure stimulation modes, adjust stimulation parameter values, and store waveforms and settings.

The systems typically further comprise an EMG sticker electrode which can be placed on the skin over any functional muscle in the body to provide a non-invasive means for the patient or physician to use EMG input from any muscle in the body to be delivered to the IPG via short-range RF telemetry. The EMG sticker electrode provides an alternative input source to the implanted EMG sensing lead.

Referring to FIG. 2, an alternative system of the present invention includes an implantable receiver stimulator coil (RSC) 20 connected to an implantable multi-channel cuff electrode (MCE) 24 by a stimulation lead. The RSC is typically MR compatible. The surgeon implants the RSC 22 subcutaneously below the clavicle in the upper chest in the axilla or in the arm and connects to the stimulation lead. The MCE 24 typically comprises a cuff having a 2-mm to 6-mm diameter and a 1-cm to 2-cm length. The cuff electrode contacts are arranged in two or more “rings” with reach ring containing four individual 2-mm×1-mm rectangular (tripolar) platinum or 90/10 platinum/iridium contacts embedded in silicone positioned at 0, 90, 180, and 270 degrees around the ring. The contacts have a 1-mm to 5-mm spacing with a 1-mm space from contact to cuff edge. This arrangement allows for monopolar stimulation of discrete neural locations as well as bipolar stimulation between two contacts. The cuff can optionally have embedded sutures in the silicone to facilitate cuff placement. The surgeon typically positions the MCE around a patient's musculocutaneous nerve and connects the connector tip end of the lead to the RSC. The cuff electrodes are configured to apply electrical current that stimulates the musculocutaneous nerve to cause the arm to flex at the elbow.

In contrast to the first embodiment, the alternative system employees an external pulse generator (EPG) and external coil to power the implanted RSC. The EPG and coil are typically disposed in a housing 26 which can be located over the RSC 20, as shown by arrow 28 in FIG. 2, to align the external and internal coils. The EPG contains electronics and battery inside titanium case and comprises or is connected to an external EMG sensor and further comprises a processor or controller which is programmed with an algorithm which detects weak electromyography signals from the EMG electrode when arm flexion is attempted. The algorithm is further programmed to deliver because the EPG to deliver stimulation to one or more channels of the MCE wrapped around the musculocutaneous nerve. EMG input from the sticker electrode will be sent to the EPG, which will typically be magnetically attached externally and interface through the skin with the implanted RSC.

The alternative systems will typically further comprise a movement remote, typically a hand-held device configured to be placed on the skin over the implant and to provide a non-invasive means for the patient to activate the RSC, to adjust the stimulation parameters (within the physician prescribed limits), and to check battery status.

The alternative systems will also typically comprise a physician programmer similar to that of the first embodiment, for example comprising a tablet computer and a telemetry cable/head which communicates with the IPG through the skin via short-range radiofrequency (RF) telemetry, allowing the physician to noninvasively interrogate and configure the IPG settings. The physician programmer has the capability to monitor EMG waveforms, configure stimulation modes, adjust stimulation parameter values, and store waveforms and settings

An external EMG sticker electrode can be placed on the skin over any functional muscle in the body and provides a non-invasive means for the patient or physician to use EMG input from any muscle in the body to be delivered to the RSC. The EMG electrode can communicate with the EPG in a wired or wireless fashion.

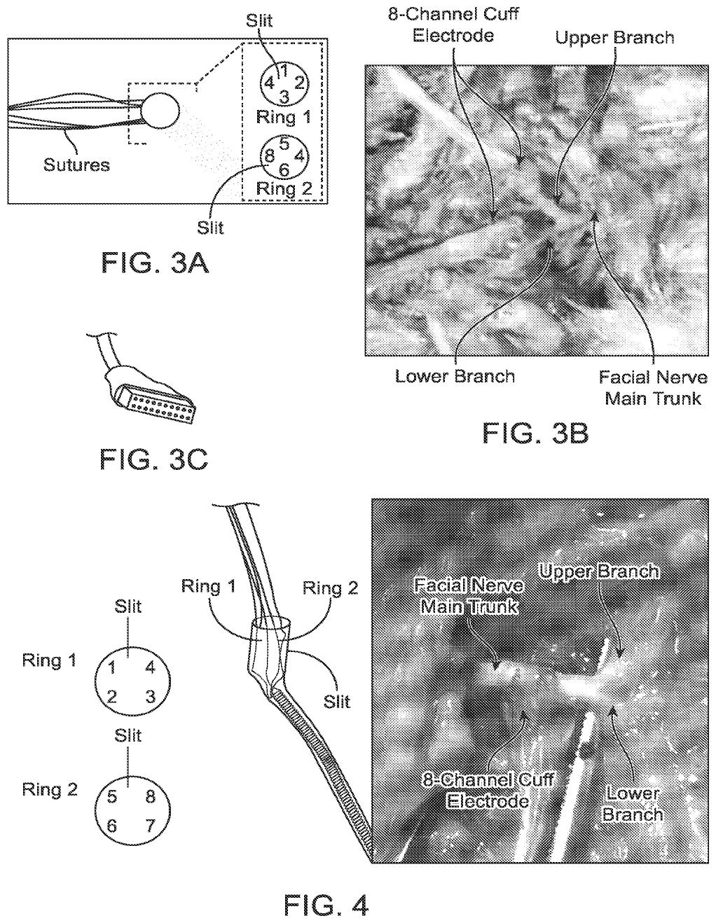

Referring to FIGS. 3 and 4, four custom 8-channel multichannel cuff electrode (MCEs) designed in accordance with the principles of the present invention were fabricated by MicroProbes for Life Science (Gaithersburg, MD, USA). Each cuff had an inner diameter of 1.5 mm, with two separate rings of four, 100 pm, rectangular, platinum contacts arranged concentrically every 90 degrees (0°, 90°, 180°, 270°) within a silicon enclosure. The arrangement of the rings and contacts allowed for monopolar stimulation of unique spatial locations on the nerve. The second parallel ring enabled field steering, in which two electrodes of the same cuff could be stimulated simultaneously to elicit an amplified response. The charge injection capacity was −164 pC/cm2 at 1 MA with a phase duration of 82 pS (0.5-1.5 KC/phase). The electrode impedance was 0.5 kfl at 1000 Hz.

The device in FIG. 4 has operational stimulation parameters that are monophasic or biphasic, with a current typically in a range from 0.1 to 20 mA, at a repetition in a range from 1 to 50 pulses per sec and duration in a range from 10 to 200 μs. The device has been designed and built to have a physician-programmable and be patient-adaptable via machine-learning capabilities in order to mimic normal use of arm flexion, for example.

The electrode structure can distribute current over any number of electrode elements or other contacts, allowing for independent and/or synchronous activation of any number of electrodes. Each electrode element or contact can be configured as the anode or cathode during the active phase of the stimulus. Stimulation parameters can be programmed, such as intensity (range 0.1 mA to 2.5 mA), pulse width (range 10 μs to 500 μs), and frequency (range 10 Hz to 50 kHz).

The stimulation waveform will typically be biphasic, asymmetric and charge balanced, with a delay of 100 μs between the active and recovery phases. Current intensity ranges from 40 μA to 2000 μA.

Apparatus of the present invention may comprise one or more cuff electrodes which can be configured to stimulate one or more nerves and/or multiple locations on a single nerve. Each cuff electrode can contain one or more contacts used to stimulate the nerve. Electrode configurations can be as simple as a single ring electrode or can be more complex with a plurality rings.

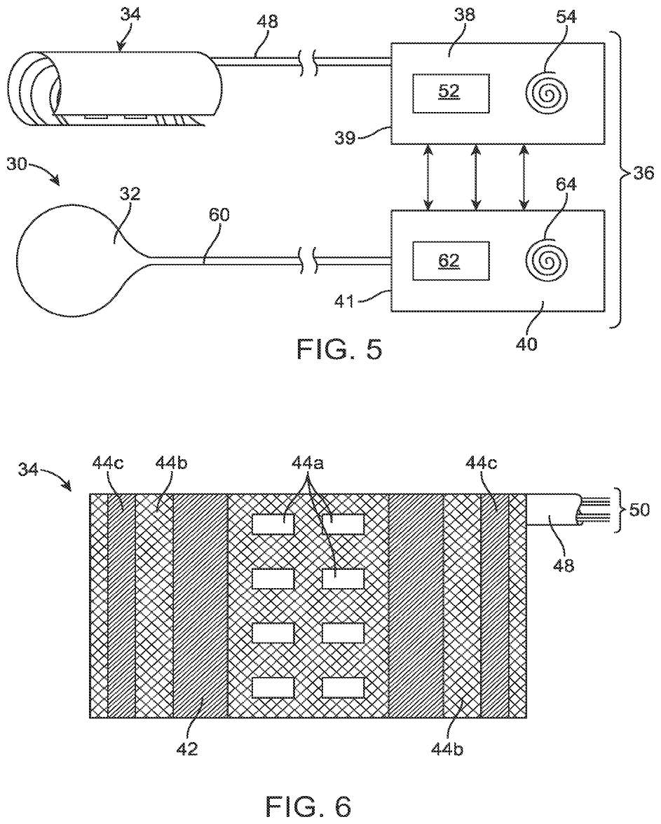

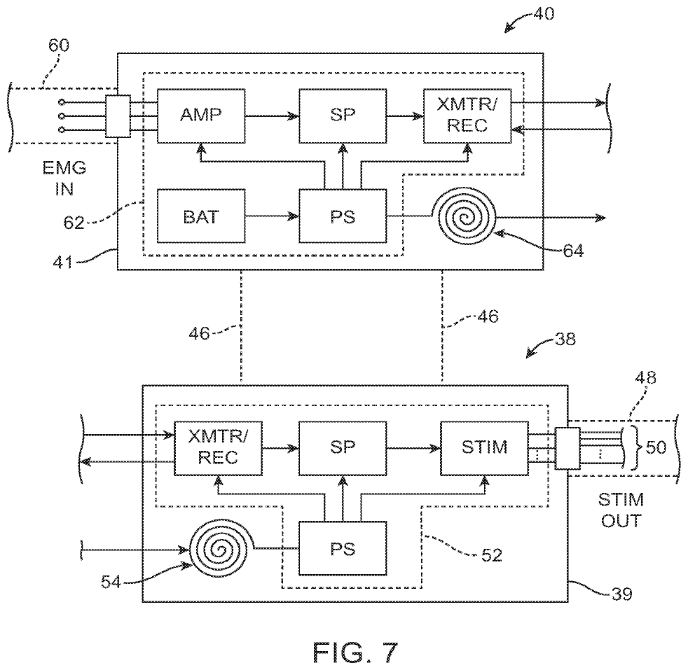

Referring now to FIG. 5, a system 30 for actuating a target muscle in accordance with the principles of the present invention will be described. The system 30 comprises an EMG sensor 32, an electrode assembly 34, and a pulse generator 36. The pulse generator 36 includes an implantable component 38 and an external component 40, where the external component is connected to the electrode assembly 34 by a lead 48 and the implantable component 40 is connected to the EMG sensor 32 by a lead 60. The implantable component 38 includes circuitry 52 and an inductive coil 54 located within an implantable housing 39, and the external component 40 includes circuitry 62 and an inductive coil 64 located within an external housing 41. The implantable housing 39 is adapted or configured to be implanted subcutaneously in any of the locations described above, such as below the clavicle in the upper chest or in the axilla, and the external housing 41 is adapted or configured to be secured to patient's skin at a location proximate the location of the implanted housing 39, preferably directly overlying the implanted housing to enhance communication between the external and implanted components of the pulse generator 36. Optimally, magnetic coupling elements (schematically indicated by broken lines 46 in FIG. 7) may be provided to help locate and immobilize the external housing 41 over the implanted housing 39.

As best scene in FIG. 6, the electrode assembly 34 preferably comprises a cuff 42, typically a multi-electrode cuff having a plurality of electrode elements 44 formed over an inner surface thereof. The electrode elements 44 are formed on an inner surface of a backing or other support matrix for the cuff 42, and the backing may be folded or rolled over a target nerve in order to engage some or all of the electrode elements against an outer surface of the nerve. The electrode elements may comprise different sizes and orientations where, for example, two, three, four or more channel electrodes 44a maybe circumferentially distributed over the inner surface of the backing so that they circumscribe the nerve when the backing is rolled or folded over the nerve. Additionally or alternatively, ring electrodes 44b may be formed to continuously circumscribe the inner surface of the cuff, and ground electrodes 44c may provided when bipolar operation is desired. Alternatively, of course, any two of the channel electrodes 44 or one or more channel electrodes and a ring electrode and/or ground may be connected to operate in a bipolar mode.

Referring now to FIG. 7, further description of the circuitry 52 and 62 in the implantable component 38 and external component 40, respectively, will be provided. The circuitry 52 in the implantable component 38 typically includes a transmitter/receiver XMTR/REC configured for transcutaneous transmission and reception of low power data (digital and/or analog) between the implantable component 38 and the external component 40. The transmitter/receiver receives power from a power supply PS which also provides power to a signal processing unit SP and a stimulator unit STIM. The power supply PS typically includes a battery or capacitor which can be recharged via inductive coil 54 which receives charge from the inductive coil 64 in the external component 40. The signal processing unit SP will be programmed to receive instructions from the external component 40 and to control and/or adjust parameters of the stimulator STIM in accordance with those instructions, The stimulator STIM, in turn, generates and selectively delivers current to the individual wires or channels 50 of the lead 48, which is connected to the cuff 34, in order to actuate the target muscle.

The circuitry 62 in the external component 40 typically includes a transmitter/receiver XMTR/REC configured for transcutaneous transmission and reception of low power data (digital and/or analog) with the implantable component 38. The transmitter/receiver receives power from a power supply PS which also provides power to a signal processing unit SP and an amplifier AMP. The power supply PS in turn is powered by a rechargeable battery that can be recharged in a wired or wireless manner as is common for handheld digital devices. Although no shown, the external component 40 will typically have a display and an I/O capability to enable programming and updating the internal logic. The amplifier AMP if configured to be externally connected to the EMG electrode 12 by lead 60, although wireless communication could also be used.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure.

Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

1.-12. (canceled)

13. A method for inducing flexion in a target muscle of a patient, said method comprising:
externally generating a drive signal;
receiving the drive signal in an external component of a pulse generator;
wirelessly transmitting a stimulation signal generated by the external component in response to the drive signal to a subcutaneously implanted component of the pulse generator; and
delivering a stimulation current produced by the implanted component of the pulse generator to a motor nerve that innervates the target muscle to induce motion of said muscle.

14. The method according to claim 13, wherein the drive signal is a flexion signal generated in response to an electromyography (EMG) signal characteristic of attempted flexion of the target muscle.

15. The method according to claim 14, wherein the target muscle comprises a patient's biceps muscle.

16. The method according to claim 14, wherein the flexion signal is produced by a patch electrode located over the target muscle at a position distal to an electrode which delivers the stimulation current to the target muscle.

17. The method according to claim 16, wherein the patch electrode is wired to the external component of the pulse generator.

18. The method according to claim 16, wherein the patch electrode is wirelessly connected to the external component of the pulse generator.

19. The method according to claim 13, wherein the stimulation signal is at least partially digital.

20. The method according to claim 13, wherein the stimulation signal is at least partially analog.

21. The method according to claim 13, wherein the stimulation current is delivered to the motor nerve by a cuff electrode wrapped around the motor nerve.

22. The method according to claim 21, wherein the cuff electrode is subcutaneously wired to the implanted component of the pulse generator.

23. The method according to claim 13, further comprising inductively recharging a battery in the implantable component of the pulse generator using a power supply in the external component.

24. The method according to claim 13, further comprising magnetically securing the external component of the pulse generator over the implanted component of the pulse generator.

25. The method according to claim 13, wherein the drive signal has a current in a range from 0.1 mA to 20 mA.

26. The method according to claim 13, wherein the drive signal has a repetition in a range from 1 pulses per sec to 50 pulses per sec.

27. The method according to claim 13, wherein the drive signal has a current in a duration in a range from 10 μs to 200 μs.
